(19) United States
(12) Patent Application Publication
(10) Pub. No.: US 2002/0122823 A1
(43) Pub. Date: Sep. 5, 2002

(54) Soft tablet containing dextrose monohydrate

(76) Inventors: Frank J. Bunick, Randolph, NJ (US); Joseph Luber, Quakertown, PA (US)

Correspondence Address: Philip S. Johnson, Esq., Johnson & Johnson, One Johnson & Johnson Plaza, New Brunswick, NJ, 08933-7003, US

(21) Appl. No.: 09/752,899

(22) Filed: Dec. 29, 2000

(51) Int. Cl. A61K 9/28; A61K 9/68; A61K 9/20; A61K 31/70

(52) U.S. Cl. 424/465; 514/23; 424/682; 514/282; 514/530; 514/569

(57) ABSTRACT

The invention relates to a tablet capable of being chewed or disintegrated in the oral cavity, which comprises a pharmaceutically active ingredient, and a matrix comprising directly compressible dextrose monohydrate and sucralose, said tablet being substantially fat free and said matrix being substantially free of non-saccharide, water soluble polymeric binders.

The present invention relates to a chewable or disintegrative tablet comprising a blend of active ingredient, dextrose monohydrate, and sucralose having exceptionally good mouthfeel and stability.

### BACKGROUND OF THE INVENTION

Pharmaceuticals intended for oral administration are typically provided in solid form as tablets, capsules, pills, lozenges, or granules. Tablets are swallowed whole, chewed in the mouth, or dissolved in the oral cavity. Chewable or disintegrative tablets are often employed in the administration of pharmaceuticals where it is impractical to provide a tablet for swallowing whole, for instance with pediatric patients. In addition, with chewable tablets, the act of chewing helps to break up the tablet particles as the tablet disintegrates and may increase the rate of absorption by the digestive tract.

Workers in the field continue to try to improve the flavor and mouthfeel of chewable tablets and other comestibles. For instance, U.S. Pat. No. 4,327,076 to Puglia et al. relates to a chewable tablet formed of particles of active ingredient isolated from other ingredients of the tablet by admixing those particles with particles formed of an edible fat or oil absorbed on a fat-absorbing material, such as microcrystalline cellulose. The particles are blended with one or more tablet bonders, such as dextrose monohydrate. In addition, the tablet may also comprise other conventional ingredients, such as sweeteners.

U.S. Pat. No.4,327,077 to Puglia et al. also relates to a chewable tablet. The tablet comprises particles of a recrystallized fatty material such as chocolate, a bulking material such as sugar or an active ingredient bound up in the particles of recrystallized fatty material, and a direct compaction vehicle that may be dextrose monohydrate.

PCT Application No. WO 99/47126 discloses compressed tablets capable of rapidly dissolving in aqueous solutions, comprising at least one non-saccharide water soluble polymer such as polyvinylpyrrolidone, optionally a saccharide of low moldability such as glucose, optionally a saccharide of high moldability such as maltose, sorbitol or a mixture thereof, and optionally a sweetener such as sucralose. These tablets are prepared by wet granulation, specifically a) granulating a formulation comprising the non-saccharide, water soluble polymer and active ingredient using no organic solvents, (b) compressing this into tablet form, (c) humidifying the tablet by exposing it to an aerated environment having at least about 50 to 100% relative humidity, and (d) drying the tablet.

As evidenced by the Puglia et al. patents, dextrose monohydrate is known to be used as a dry binder in tablets formed by direct compression methods. However, the Puglia et al. formulations include fats in order to achieve a creamy mouthfeel. Applicants have now discovered that directly compressible grades of dextrose monohydrate in particular impart a smooth, creamy texture and fast melt-away to soft tablets designed for chewing or dissolving in the mouth prior to swallowing. The addition of fat to such tablets is advantageously not required. Nor is the addition of water soluble binders required. The tablets have substantially superior mouthfeel compared to tablets containing more commonly used excipients such as mannitol, sorbitol, or standard tableting sugar. For taste, the present tablets comprise the high intensity sweetener sucralose. Conventional high intensity sweeteners, such as aspartame, when combined with dextrose monohydrate, result in age related browning or discoloration. However, applicants have also found that the combination of sucralose and directly compressible dextrose monohydrate causes no unwanted browning with aging.

### SUMMARY OF THE INVENTION

The present invention provides a tablet capable of being chewed or disintegrated in the oral cavity prior to swallowing, comprising a pharmaceutically active ingredient, and a matrix comprising directly compressible dextrose monohydrate and sucralose, said tablet containing less than 5% fat and said matrix being substantially free of non-saccharide, water soluble polymeric binders.

### DETAILED DESCRIPTION OF THE INVENTION

The tablet is made from a mixture comprising one or more active ingredients, directly compressible dextrose monohydrate, and sucralose. Suitable active ingredients include pharmaceuticals, minerals, vitamins and other nutraceuticals. Suitable pharmaceuticals include analgesics, decongestants, expectorants, antitussives, antihistamines, gastrointestinal agents, diuretics, bronchodilators, sleep-inducing agents and mixtures thereof Preferred pharmaceuticals for use as the active ingredient include acetaminophen, ibuprofen, flurbiprofen, naproxen, diclofenac, aspirin, pseudoephedrine, phenylpropanolamine, chlorpheniramine maleate, dextromethorphan, diphenhydramine, famotidine, loperamide, ranitidine, cimetidine, astemizole, terfenadine, fexofenadine, cetirizine, antacids, mixtures thereof and pharmaceutically acceptable salts thereof. More preferably, the active ingredient is selected from the group consisting of acetaminophen, ibuprofen, pseudoephedrine, dextromethorphan, diphenhydramine, chlorpheniramine, calcium carbonate, magnesium hydroxide, magnesium carbonate, magnesium oxide, aluminum hydroxide, mixtures thereof, and pharmaceutically acceptable salts thereof.

The active ingredient(s) are present in the tablet in a therapeutically effective amount, which is an amount that produces the desired therapeutic response upon oral administration and can be readily determined by one skilled in the art. In determining such amounts, the particular active ingredient being administered, the bioavailability characteristics of the active ingredient, the dose regime, the age and weight of the patient, and other factors must be considered, as known in the art.

The active ingredient can be in the form of a fine powder, granule, or large crystal, and typically has an average particle size from about 20 to about 1000 microns. Preferably the active ingredient has an average particle size from about 50 to about 700 microns, and more preferably the active ingredient has an average particle size from about 500 to about 500 microns.

If the active ingredient has an objectionable taste, it may be coated with a taste masking coating, as known in the art. Examples of suitable taste masking coatings are described in U.S. Pat. No. 4,851,226, U.S. Pat. No. 5,075,114, and U.S. Pat. No. 5,489,436. Commercially available taste masked active ingredients may also be employed. For example, acetaminophen particles which are encapsulated with ethylcellulose or other polymers by a coaccervation process may be used in the present invention. Coaccervation-encapsulated acetaminophen may be purchased commercially from Eurand America, Inc. Vandalia, Ohio, or from Circa Inc., Dayton, Ohio.

The active ingredient is contained in a matrix comprising dextrose monohydrate and sucralose. The dextrose monohydrate is present in the tablet in directly compressible form. That is, the dextrose monohydrate has an average particle size of about 100 to about 500 microns, preferably about 100 to about 250 microns, more preferably about 150 to about 200 microns. Such a particle size is required to impart the formulation with adequate flowability and compressibility, and with a smooth and creamy mouthfeel according to the invention.

The amount of dextrose monohydrate in the tablet is typically about 15 to about 90% by weight, preferably about 25 to about 85% by weight, and more preferably about 30 to about 75% by weight of the total weight of the tablet.

The use of directly compressible dextrose monohydrate at these levels enables the minimization or elimination of cellulosic dry binders such as microcrystalline cellulose from the formula. The avoidance of microcrystalline cellulose improves both the taste and the mouthfeel of the resulting tablets. While it may be desirable to use microcrystalline cellulose at relatively low levels in the formulation for its disintegrant properties, the higher levels generally used for binding properties are not necessary. The amount of microcrystalline cellulose in the tablet is preferably less than about 20% by weight, more preferably less than about 10% by weight of the tablet, and most preferably, the tablet is substantially free of microcrystalline cellulose.

The use of directly compressible dextrose monohydrate at these levels also enables the minimization or elimination of fat. Fats are generally understood to mean esters of glycerol and fatty acids, which can include monoglycerides, diglycerides, and triglycerides. Preferably, the tablet of the present invention contains less than about 5% fat. More preferably, the tablet contains less than about 3% by weight of fat, most preferably the tablet contains substantially no fat. Fat-free formulations are more stable at elevated temperatures, eliminating the need for specially controlled shipping and storage conditions. Additionally, fats are susceptible to oxidative and chemical hydrolysis, leading to a “rancid” taste and/or odor. This effectively shortens the shelf-life of a product. Preferably the melting point of any fats or other oily materials that are included in the composition is greater than about 80° F. in order to maintain product stability at elevated temperatures during shipping or storage.

In a particularly preferred embodiment of the invention the tablet is substantially free of tri-glycerides specifically. Triglycerides are more hydrophobic than mono- and di-glycerides, and are expected to hinder dissolution of the active ingredient. This is undesirable in an immediate release dosage form such as the tablet of this invention.

Sucralose, 4,1′6′-trichloro-4,1,6′-trideoxy-galactosucrose, is a high intensity sweetener manufactured from sucrose as a starting material. This and other chlorine-substituted sucrose sweeteners are disclosed in British Patent No. 1,544,167, and in British Patent Application No. 2,104,063A.

The amount of sucralose in the tablet is typically about 0.005 to about 10% by weight of the total weight of the tablet, preferably about 0.01 to about 5% by weight, and more preferably about 0.5 to about 2% by weight of the total weight of the tablet.

Preferably, the weight ratio of dextrose monohydrate to sucralose in the tablet is at least about 25:1, more preferably at least about 50:1, most preferably at least about 75:1.

The tablet may contain other conventional ingredients, such as fillers, conventional dry binders, other sweeteners, disintegrants, and lubricants. The mixture may also incorporate pharmaceutically acceptable adjuvants, including, for example, preservatives, flavors, acidulants antioxidants, glidants, surfactants, and coloring agents. However, the tablet preferably comprises no more than about 25 weight % of such optional auxiliary ingredients.

The tablet may be made in any manner, and a variety of tableting methods are known in the art. Conventional methods for tablet production include direct compression (“dry blending”), dry granulation followed by compression, and wet granulation followed by drying and compression. Other methods include the use of compacting roller technology such as a chilsonator or drop roller, or molding, casting, or extrusion technologies. All of these methods are well known in the art, and are described in detail in, for example, Lachman, et al., The Theory and Practice of Industrial Pharmacy, Chapter 11, (3^{rd} Ed. 1986).

Preferably the tablets are formed by the direct compression method, which involves directly compacting a blend of the active ingredient, dextrose monohydrate, sucralose, and any other appropriate optional ingredients. After blending, a pre-determined volume of particles is filled into a die cavity of a rotary tablet press, which continuously rotates as part of a “die table” from the filling position to a compaction position. The particles are compacted between an upper punch and a lower punch to an ejection position, at which the resulting tablet is pushed from the die cavity by the lower punch and guided to an ejection chute by a stationary “take-off” bar. The direct compression process enables the minimization or elimination of water-soluble, non-saccharide polymeric binders such as polyvinyl pyrrolidone, alginates, hydroxypropyl cellulose, hydroxypropylmethylcellulose, hydroxyethylcellulose, and the like, which can have an adverse effect on dissolution.

Preferably, tableting is carried out such that the tablet is relatively soft. The hardness of the tablet is preferably up to about 15 kiloponds per square centimeter (kp/cm²). More preferably, the hardness of the tablet is in the range of about 1 to 8 kp/cm², most preferably about 2 to 6 kp/cm². Hardness is a term used in the art to describe the diametral breaking strength as measured by conventional pharmaceutical hardness testing equipment, such as a Schleuniger Hardness Tester. In order to compare values across different size tablets, the breaking strength is normalized for the area of the break (which may be approximated as tablet diameter times thickness). This normalized value, expressed in kp/cm², is sometimes referred in the art as tablet tensile strength. A general discussion of tablet hardness testing is found in Leiberman et al., Pharmaceutical Dosage Forms—Tablets, Volume 2, 2^{nd} ed., Marcel Dekker Inc., 1990, pp. 213-217, 327-329.

Specific embodiments of the present invention are illustrated by way of the following examples. This invention is not confined to the specific limitations set forth in these examples, but rather to the scope of the appended claims. Unless otherwise stated, the percentages and ratios given below are by weight.

### EXAMPLES

Two batches of tablets were made and taste tested for smoothness, dryness, grittiness, and overall liking. Tablet 1 was made according to the invention and Tablet 2 was comparative:

Tablet 1

| Ingredient | mg/tab |
|---|---|
| Sucralose | 8.0 |
| FD&C Yellow #6 Al Lake | 3.0 |
| Orange Flavor | 10.0 |
| Crospovidone NF | 15.0 |
| Coated Ibuprofen Gran | 140.6 |
| Dextrose Monohydrate | 850.0 |
| Magnesium Stearate NF | 7.5 |

Tablet 2

| Ingredient | mg/tab |
|---|---|
| Sucralose | 8.0 |
| FD&C Yellow #6 Al Lake | 3.0 |
| Orange Flavor | 10.0 |
| Crospovidone NF | 15.0 |
| Coated Ibuprofen Gran | 140.6 |
| Mannitol | 850.0 |
| Magnesium Stearate NF | 7.5 |

Both tablets were compressed using 9/16″ biconcave tooling to a hardness of 3 to 4 kp.

Nine people evaluated the two samples. Approximately half evaluated Tablet 1 first, then Tablet 2, the others evaluated Tablet 2 then Tablet 1. Samples were evaluated for smoothness, dryness, grittiness, and overall liking. The mean values for each sample were:

| | Mean for Tablet 1 | Mean for Tablet 2 |
|---|---|---|
| | (n = 9) | (n = 9) |
| Smoothness | 4.11 | 3.11 |
| (1 = not smooth, 5 = very smooth) | | |
| Dryness | 2.00 | 3.44 |
| (1 = not dry, 5 = very dry) | | |
| Grittiness | 1.56 | 2.11 |
| (1 = not gritty, 5 = very gritty) | | |
| Overall Liking | 4.06 | 3.00 |
| (1 = dislike, 5 = like a lot) | | |

Based on a statistical analysis, the following can be concluded:

1. Tablet 1 containing dextrose monohydrate was smoother than Tablet 2 containing mannitol.

2. Tablet 1 containing dextrose monohydrate was not as dry as Tablet 2 containing mannitol.

3. Tablet 1 containing dextrose monohydrate was not as gritty as Tablet 2 containing mannitol.

4. Tablet 1 containing dextrose monohydrate was preferred overall over Tablet 2 containing mannitol.

We claim:

1. A tablet capable of being chewed or disintegrated in the oral cavity prior to swallowing, comprising a pharmaceutically active ingredient, and a matrix comprising directly compressible dextrose monohydrate and sucralose, said tablet containing less than 5% fat and said matrix being substantially free of non-saccharide, water soluble polymeric binders.

2. The tablet of claim 1, wherein the active ingredient is selected from the group consisting of acetaminophen, ibuprofen, pseudoephedrine, dextromethorphan, diphenhydramine, chlorpheniramine, calcium carbonate, magnesium hydroxide, magnesium carbonate, magnesium oxide, aluminum hydroxide, mixtures thereof, and pharmaceutically acceptable salts thereof.

3. The tablet of claim 1, wherein the directly compressible dextrose monohydrate has an average particle size of about 100 to about 250 microns.

4. The tablet of claim 1, wherein the weight ratio of dextrose monohydrate to sucralose is at least about 25:1.

5. The tablet of claim 1 containing about 15 to about 90% by weight of dextrose monohydrate based on the total weight of the tablet.

6. The tablet of claim 1 containing about 0.005 to about 10% by weight of sucralose based on the total weight of the tablet.

7. The tablet of claim 1 containing less than 3% by weight fat.

8. The tablet of claim 1 being substantially free of aspartame.

9. The tablet of claim 1 wherein the pharmaceutically active ingredient has an average particle size from about 100 to about 500 microns.

10. The tablet of claim 1 manufactured by a direct compression or dry granulation process.

11. The tablet of claim 1 being substantially free of microcrystalline cellulose.

12. A tablet capable of being chewed or disintegrated in the oral cavity prior to swallowing, comprising a pharmaceutically active ingredient; and a matrix comprising directly compressible dextrose monohydrate; sucralose; at least one disintegrating agent selected from microcrystalline cellulose, starch, sodium starch glycolate, crosslinked polyvinylpyrrolidone, crosslinked carboxymethylcellulose, and mixtures thereof; at least one lubricant selected from magnesium stearate, stearic acid, and mixtures thereof; and optionally an auxiliary ingredient selected from fillers, sweeteners, surfactants, glidants, acidulents, antioxidants, preservatives, coloring, flavoring agents, and mixtures thereof; said tablet being substantially free of triglycerides and said matrix being substantially free of non-saccharide, water soluble polymeric binders.

13. The tablet of claim 13 wherein the tablet comprises no more than 25% by weight of said optional auxiliary ingredients.
